(19) United States
(12) Patent Application Publication
(10) Pub. No.: US 2004/0076564 A1
(43) Pub. Date: Apr. 22, 2004

(54) Multi-layer products having improved strength attributes

(76) Inventors: Lisa A. Schild, Roswell, GA (US); Jeffrey L. McManus, Canton, GA (US)

Correspondence Address: Mr. Stephens E. Bondura, DORITY & MANNING, P.A., One Liberty Square, 55 Beattie Place, Suite 1600, Greenville, SC, 29601, US

(21) Appl. No.: 10/272,544

(22) Filed: Oct. 16, 2002

(51) Int. Cl. A61L 2/20

(52) U.S. Cl. 422/292

(57) ABSTRACT

A single step sterilization wrapping material suitable for use, as an example, in wrapping surgical instruments and supplies for sterilization, transportation and storage, is provided. The material includes separate first and second wraps that provide improved strength attributes to the system. Separate wraps are provided that may be specially engineered to deliver selected properties for each sheet and then joined to one another to permit wrapping and opening in a neat single step process which increases efficiency and saves time and money. The sheets may be joined in a manner that increases the strength and/or tear resistance of the sterilization wrapping material.

### BACKGROUND OF THE INVENTION

The present invention is directed to multi-layer products. More particularly, the present invention is directed to multi-layer materials made from individual sheets that are joined to one another so as to be suitable for use as a sterilization wrap for wrapping surgical instruments and supplies for sterilization and storage in conjunction with surgical procedures and for other applications such as packaging items for bone marrow units.

Personnel in the Central Service Room (CSR) or the Sterile Processing Department (SPD) of hospitals are commonly charged with the responsibility of packaging surgical supplies to ensure that the sterility of the packaged contents are maintained all the way from sterilization to the point of reuse. Several activities are involved in the task of sterile supply delivery to the operating room and other units.

Much of the surgical instruments and supplies used in the operating room are reusable. These supplies typically include such things as clamps, scalpel blade handles, retractors, forceps, scissors, surgeons towels, basins and the like. All of these supplies must be collected after each procedure and sterilized before they may be used again in another procedure. To this end, the supplies are placed in stainless steel instrument trays, and soft goods such as surgeon's towels, drapes, and gowns are prepared for packaging. Then, the trays and package contents may be wrapped with two sheets of material commonly referred to as sterilization wrap.

The sterilization wrap is usually a sheet of woven or nonwoven material that when wrapped around the tray or package contents in a certain prescribed manner will permit the entry of sterilizing vapor/gas or other medium to sterilize the contents of the tray while denying the ingress of contaminants such as bacteria and other infectious causing materials or their vehicles after sterilization. The sterilization wrap may be used to sterilize by wrapping the item and subjecting the wrapped item to a sterilization technique. Two primary sterilizing techniques for sterilizing instruments are autoclaving with steam and ethylene oxide sterilization.

Using a wrapped tray as an example, once the wrapped tray and its contents have been sterilized, the wrapped tray is transported to the point of use, typically an operating room, or is stored until it is ready to be used. During storage and transfer to the operating room, the wrapped tray may be handled several different times. Each time the wrapped package is handled, there is a potential that the sterile nature of the package contents may be compromised. The two most common ways the wrapped package may be compromised are a tear or other breach of the sterilization wrap, and wetness or foreign materials identified on the outer sheet of the sterilization wrap, which would warrant a premature unwrapping.

To promote and maintain the sterility of the packaged contents, the Association of Operating Room Nurses (AORN) has developed certain recommended practices for the wrapping and handling of in-hospital processed packages. It is common practice among many hospitals as recommended by the AORN to “double wrap” in-hospital processed packages. A primary method of double wrapping is “sequential” in nature in that the package contents are first wrapped by one sheet of sterilization wrap and then wrapped again by another sheet of sterilization wrap. Another method of double wrapping is “simultaneous” in nature in that the package contents are wrapped by two sheets of sterilization wrap at the same time. That is, two sheets of sterilization wrap are aligned one on top of the other, and the item to be wrapped is placed on top of the two sheets, then the item is wrapped by both sheets of material at the same time.

Studies have been used to track packages from initial wrapping, all the way through sterilization, storage, handling, transfer, unwrapping and ultimate reuse. These studies indicate that the frequency of compromising wrapped items due to tears or holes has been reduced because of improved handling and storage techniques and because of improved sterilization packaging products. One of the main thrusts behind such efforts has been economics. Every time a sterile package is compromised, it must be taken out of circulation, unwrapped, rewrapped, and resterilized before it may properly be reused. This process wastes time and money.

While the frequency of compromising wrappers has been reduced, thus resulting in the saving of time and money, the use of simultaneous wrapping techniques would further increase the time savings in wrapping and opening packages and thus result in a still greater cost savings. Simultaneous wrapping takes less time than sequential wrapping, and recent research in hospitals has shown simultaneous wrapping to be just as effective as sequential wrapping in maintaining sterility absent a breach in the wrap that may be independent of the manner of wrapping.

Even though the hospital staff may want to simultaneously wrap instead of sequentially wrap, the time it takes to set up the outer and inner sheets of sterilization wrap and the awkwardness of manipulating loose sheets during simultaneous wrapping may offset the time savings hoped to be achieved when attempting to move away from sequential wrapping. Consequently, if a product existed which provided the appropriate inner and outer sheet combinations and eliminated the awkwardness of keeping the two sheets together during the package wrapping and opening processes, then a simultaneous packaging system would deliver one or more benefits including, but not limited to, time savings and/or selected inner and outer sheet performance design parameters.

In conjunction with the manner in which the packages are wrapped, the material used for wrapping is also important. As mentioned above, the two most common wrapping materials are woven materials such as cloth (cotton/polyester), nonwoven materials such as KIMGUARD® Sterile-Wrap (polypropylene) from Kimberly-Clark Corporation of Neenah, Wisconsin and Bio-shield CSR Wrap (wood pulp/polyester) from Baxter Healthcare Corporation of Deerfield, Illinois. One version of the Baxter sterilization wrap is a product called DualWrap® Sterilization Wrap, which includes an inner sheet of wet laid paper (cellulose) and a separate outer sheet of spunlaced or hydroentangled pulp/polyester. The inner and outer sheets are provided in a stack of loose, unattached sheets in which the inner and outer sheets are alternated.

Whatever the material is that is being used as sterilization wrap, it should be noted that when wrapping two sheets at the same time, it is important that the wrapping materials provide good barrier properties to maintain package sterility and/or good strength properties so that tearing or other forms of breaching are held to a minimum. Consequently, there is a need for a new sterilization wrap that actually reduces the time for packaging and opening and/or provides increased strength and tear resistance versus currently used sterilization wraps. Such attributes are provided by the present invention as will become more apparent upon a further review of the following specification, claims and drawings.

### SUMMARY OF THE INVENTION

The present invention provides a sterilization wrap for wrapping items in packages which are to be sterilized and maintained in a sterilized condition until use such as surgical instruments for hospital operating room use. A large number of such items are currently wrapped by two separate sheets of sterilization wrap. The most common method of wrapping such items is called double, sequential wrapping wherein an item is wrapped in a first sheet of sterilization wrap with the loose ends being taped shut. Next, a second and separate sheet of sterilization wrap is used to wrap the item a second time. Once the second sheet of wrap has been wrapped around the item, the loose ends of the second sheet are taped closed and the wrapped item is sent through a sterilization process. After the wrapped item has been sterilized, it is normally placed in storage until actual use at which time the wrapped and sterilized package is removed from storage and transported to the operating room where the sterilization wrap is removed and the items are subsequently used. A second and less commonly used method of wrapping is called the simultaneous wrapping wherein two sheets of sterilization wrap are placed one on top of the other, aligned and then the two sheets are wrapped about the item to be sterilized at the same time. After wrapping is complete, the loose ends are taped shut and the item is sent through the same sterilization process as described above.

The present invention provides a single-step system for simultaneously wrapping and unwrapping items that must be sterilized prior to use. This is accomplished by bonding or joining two separate sheets of sterilization wrap together at one or more bond points to create a single step system wherein the separate sheets are pre-aligned and joined to one another to facilitate the wrapping process as well as the unwrapping process. As a result, the amount of time needed to wrap and unwrap an item is decreased and the ease of wrapping is improved. In addition, each of the individual sheets of the sterilization wrap may be engineered or designed to impart special or different features to the overall system. The sheets are also pre-aligned to increase the overall strength of the wrap system such that system is better able to withstand in-use handling conditions.

The single step sterilization wrap includes a first sheet comprising a nonwoven material having a majority of fibers oriented in a direction parallel, or substantially parallel, to one side of the sheet; a second sheet comprising a nonwoven material having a majority of fibers oriented in a direction parallel, or substantially parallel, to one side of the sheet; wherein the second sheet is rotated and joined to the first sheet at one or more bond points such that the majority of oriented fibers in the second sheet are substantially orthogonal to the majority of oriented fibers in the first sheet. As used herein, the term “sheet” is meant to include single-layer materials, such as a woven or nonwoven fabric, and multi-layer materials, such as laminates. The individual first and second sheets may be made from a variety of sterilization materials, including fibrous materials such as nonwovens and wovens. The first sheet includes fibers substantially oriented in a direction parallel, or substantially parallel, to one side of the sheet and the second sheet includes fibers substantially oriented in the same direction, but wherein the second sheet is rotated such that the oriented fibers are substantially orthogonal to those in the first sheet. As a result, when the first and second sheets are joined, the sterilization wrap has more uniform strength properties, thereby increasing the overall strength of the system.

The present invention also provides methods of making sterilization wraps by taking a first sheet comprising a nonwoven material having a majority of fibers oriented in a direction parallel, or substantially parallel, to one side of the sheet; taking a second sheet comprising a nonwoven material having a majority of fibers oriented in the same direction; rotating the second sheet such that the majority of oriented fibers in the second sheet are substantially orthogonal to the majority of oriented fibers in the first sheet; and joining the first sheet to the second sheet at one or more bond points. The second sheet may be rotated from about 90, or from about 60 to about 90 degrees relative to the first sheet.

The sterilization wrap has a first exterior surface and a second exterior surface formed by the opposed sides of the system with each of the surfaces having respective surface area and wherein the bond points joining the first and second sheets together occupy no more than 50% of the surface area of either the first or second exterior surfaces of the sterilization wrap. The first and second sheets may be joined to one another in a variety of bonding patterns including both long continuous bonds and point bonding. In one embodiment, the sterilization wrap may define a first zone and a second zone with the first zone having a greater number of the bond points than the second zone and wherein the second zone is surrounded by the first zone so that the sterilization wrap has an area of low density bonding points surrounded by an area of higher density bonding points. In another embodiment, the first zone is surrounded by the second zone so that the sterilization wrap has an area of higher density bonding points surrounded by an area of lower density bonding points.

Each of the individual sheets may be designed to have particular properties which may be the same or different from the other sheet of the sterilization wrap of the present invention. For example, the second sheet may be made stronger than the first sheet as indicated by the second sheet having a greater grab tensile strength as compared to the first sheet. In addition, the barrier properties of the first sheet may be fortified to create a better means of filtering bacteria than the second sheet.

The first sheet and second sheet may both be made from nonwoven laminates such as spunbond/meltblown/spunbond laminates wherein the inner meltblown layer provides barrier properties and the outer spunbond layers provides strength. By using a heavier basis weight meltblown layer in the first sheet as compared to the second sheet, the first sheet will have a better barrier property than the second sheet in which case the first sheet will have a lower dry spore penetration rate than the second sheet and a greater bacterial filtration efficiency than the second sheet. Conversely, the meltblown layer of the first sheet may be decreased to such an extent that the bacterial filtration efficiency of the first sheet is less than the second sheet. Furthermore, the strength of the first and second sheets may be varied by varying the basis weight and the types of polymers being used to form the fibers which make up the individual layers of the respective laminates. As a result, a sterilization wrap may be designed wherein the peak energy of the second sheet is greater than the first sheet.

The present invention provides, however, that regardless of the different properties for each of the first and second sheets, the first sheet be positioned such that the majority of fibers are substantially oriented in one direction and the second sheet be positioned such that the oriented fibers are substantially orthogonal to the oriented fibers in the first sheet such that, when joined, the sterilization wrap has more uniform strength properties, thereby increasing the overall strength of the system as compared to prior art wrap systems.

### BRIEF DESCRIPTION OF THE DRAWINGS

FIG. 1 is a perspective view of a single step sterilization wrap according to the present invention with a sterilization tray ready for wrapping placed on top of the sterilization wrap.

FIG. 2 is a cross-sectional side view of a single step sterilization wrap according to the present invention.

FIGS. 3 through 6 are top plan views of additional embodiments of single step sterilization wraps according to the present invention with different bonding patterns for joining the separate sheets of the sterilization wrap together.

### DETAILED DESCRIPTION OF THE INVENTION

Disclosed herein is a sterilization system suitable for use with simultaneous wrapping procedures for wrapping, sterilizing, storing and using sterilized items such as surgical supplies. While the present invention will be described in conjunction with its use in hospital and surgical room procedures, the sterilization system of the present invention may be used wherever there is a need for sterilized materials. Consequently, the following description of the present invention should not be considered a limitation of the scope of use of the present invention.

The present invention provides a sterilization wrap having higher strength and tear resistant attributes as compared to known sterilization wraps. As such, the present invention is better able to withstand in-use handling conditions, which may create abrasions, cuts or holes in the wrap product.

In previous single sheet wrap systems, nonwoven webs are produced having substantially non-uniform strength properties. This non-uniformity is caused by the fiber laydown behavior at high speeds, resulting in a larger percentage of oriented fibers. As a result, these previous wrap systems have increased strength attributes in a direction parallel, or substantially parallel, to one side of the sheet, or, in this example, the Machine Direction (MD) of the web. However, as MD strength increases, these nonwoven webs become more susceptible to strength-related failures in the orthogonal direction, or, in this example, the Cross Direction (CD) of the web. Multi-layer nonwoven sheets also have non-uniform MD and CD strength properties since they are often joined to one another with fiber alignment in the MD.

As such, the present invention optimizes the overall strength of the sterilization wrap by rotating one sheet of the sterilization wrap such that the fibers in the first sheet are substantially oriented in direction parallel, or substantially parallel, to one side of the sheet and the oriented fibers in the second sheet are substantially orthogonal to the oriented fibers in the first sheet. As a result, the fiber orientation will produce a sterilization wrap product having more uniform strength properties, such that the overall product strength is increased.

To optimize strength, the sheets may be joined in a substantially “90-degree sheet-to-sheet” orientation. As used herein, a “90-degree sheet-to-sheet” orientation describes a product wherein both sheets have a larger percentage of fibers oriented in the MD and the second sheet is rotated 90 degrees relative to the first sheet and then joined to the second sheet such that the first sheet now has a larger percentage of fibers oriented substantially orthogonal to the fibers in the second sheet, which has larger percentage of fibers oriented orthogonal to the MD. However, it should be understood that while one embodiment rotates the second sheet 90 degrees, other embodiments may be used such that the second sheet is rotated at angles less than about 90 degrees. These embodiments may be used and the actual angle of rotation may depend on one or more factors, including, but not limited to, the nonwoven material(s) used, the selected strength of the system in the MD, the selected strength of the system in the CD, the degree of uniformity of the CD and MD strengths in the finished product, and/or the percentage of MD oriented fibers in each sheet prior to rotation. The present invention contemplates embodiments wherein the second sheet is rotated from about 90, or from about 60 to about 90 degrees relative to the first sheet.

In one embodiment of the present invention, the sterilization wrap uses two sheets of spunbond/meltblown/spunbond (SMS) laminates. The two sheets are joined to one another at one or more bond points. The multi-layer construction enables simultaneous wrapping, which may be beneficial in a hospital environment.

The present invention will now be described by reference to drawings showing different embodiments of the present invention. It is to be understood that these embodiments are non-limiting and that other embodiments are contemplated by the present invention.

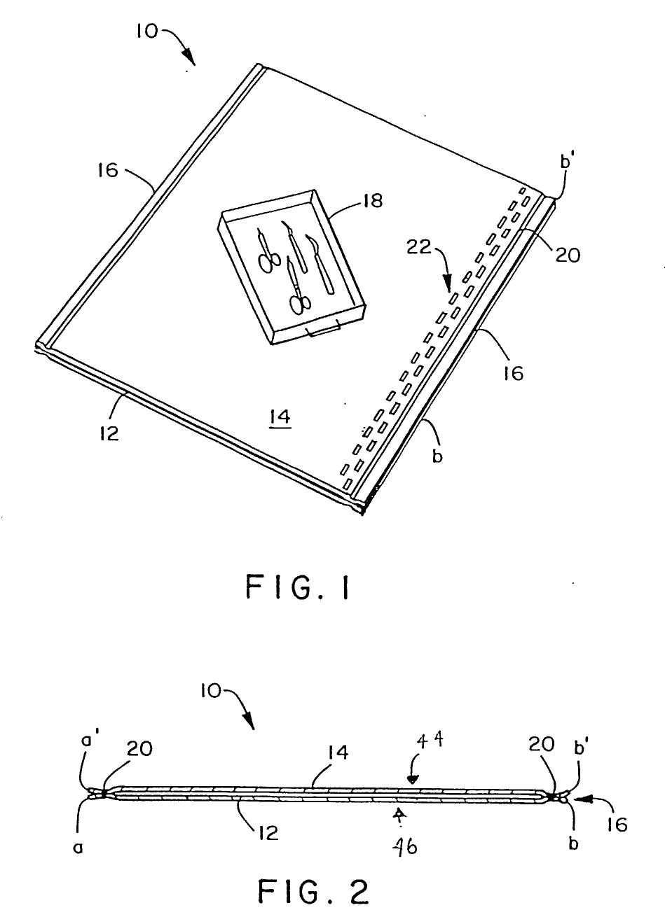

Referring to FIGS. 1 and 2 of the drawings, there is shown a sterilization system or wrap for containing and maintaining sterility of surgical supplies and the like. The sterilization wrap 10 includes a second sheet 12, which may be referred to as a strength reinforced barrier web laminate, and a first sheet 14, also referred to as a barrier web laminate. As may be seen from FIG. 1, the second sheet 12 and first sheet 14 are placed in face to face relationship with one another, one on top of the other in vertical juxtaposition. Each of the sheets may be of substantially the same size and shape. In one embodiment, the sheets will be square or rectangular in shape. As a result, each sheet will have at least two relatively parallel edges a,a′ and b,b′ located about their peripheries 16. The sheets are oriented in a substantially 90 degree sheet-to-sheet orientation such that the second sheet 12 has fibers substantially oriented in the MD and the first sheet 14 has fibers substantially orthogonal to the fibers of the second sheet 12.

To facilitate wrapping of an item 18 such as is shown in FIG. 1, the second sheet 12 and the first sheet 14 are attached to one another in a manner so as to hold the two sheets together. The two sheets may be joined about all or a portion of their peripheries 16. As used herein, the periphery of the first sheet and the second sheet is meant to include that portion of each wrap from an edge of the sheet and the surface area immediately adjacent thereto. As shown in FIGS. 1 and 2, the two sheets are joined to one another along the entire length of two substantially parallel edges of each sheet, a-a′ and b-b′ . The edges may be joined to one another by any number of suitable means including, but not limited to, adhesives, stitching, heat bonding and ultrasonic bonding collectively referred to as joining. As shown in FIGS. 1 and 2, the bond points 20 may be perfected by ultrasonic bonding, may be continuous, and may run the entire length of the edges just interior to or along the edge and/or the periphery 16 on opposed sides of the sheets 12 and 14. Alternative embodiments include point bonds on adjacent sides of the sheets 12 and 14; and continuous and/or point bonds on three sides or all four sides and/or edges of the sheets 12 and 14.

In addition to or as an alternate to the continuous bonds 20, a second set of bond points 22 may be used to secure the two sheets together. The bond points 22 in FIG. 1 are a series of spaced-apart and separate bond points in the form of two rows of parallel but spaced apart rectangles or other shapes with the rectangles in one row being offset from the other row so that they are in overlapping relationship if the system 10 were viewed edge on. This type of bond point pattern has been used to seam sleeves on disposable surgical gowns manufactured by the assignee of record, Kimberly-Clark Corporation of Neenah, Wisconsin. The bond points 22 may be just interior of the continuous bond points 20 and serve to further join the two sheets 12 and 14 together when used alone or in conjunction with the continuous bond poins 20.

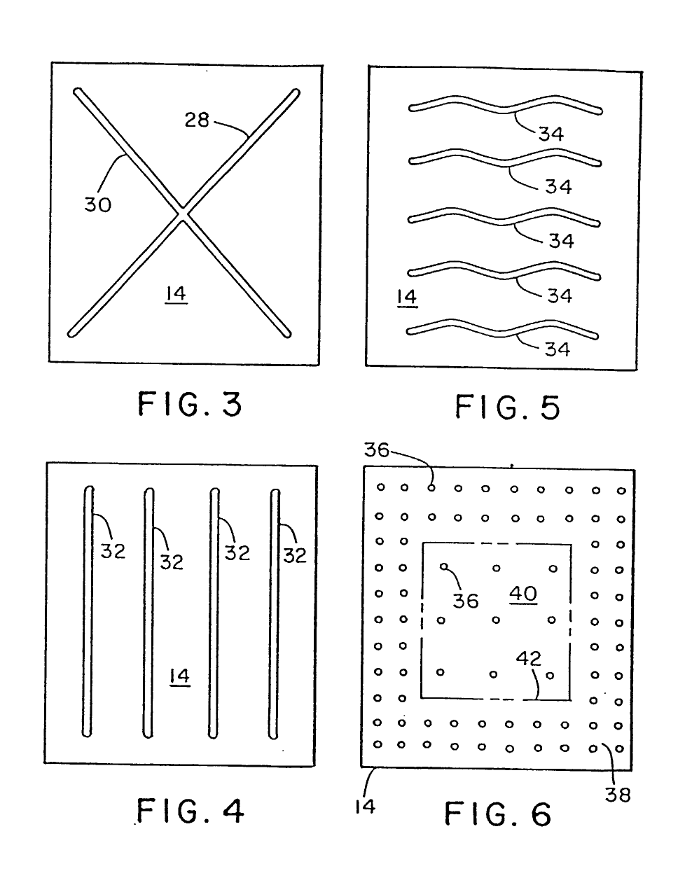

It also is possible to effect bonding between the two sheets 12 and 14 in a variety of other manners which are exemplified, at least in part, in FIGS. 3 through 6. In FIGS. 3 through 6, the first and second sheets are superposed and joined to one another by one or more bond points that may be long continuous bond lines, such as are shown in FIGS. 3 through 5, or a plurality of localized bond points, such as are shown in FIG. 6. In FIG. 3, which is a top plan view, the second sheet 12 and first sheet 14 of the system 10 are bonded together by two crisscrossing bond point lines 28 and 30 which form an “X”-pattern across the surface of the system 10. In FIG. 4, the second sheet 12 and the first sheet 14 of the system 10 are bonded to one another by a series of substantially parallel bond points 32 that span all or a portion of the length or width of the system 10. In FIG. 5, a series of substantially sinusoidal bond points 34 are provided.

In addition to, or in conjunction with, the relatively long bond points or seams shown in FIGS. 3 through 5, the second sheet 12 and the first sheet 14 of the system 10 may be joined by a plurality of localized, discontinuous bond points 36, such as are shown in FIG. 6. These bond points may be uniformly spaced across the surface of the system 10, or they may be broken into two or more zones with each of these zones having varying degrees or densities of bond points. Referring to FIG. 6, the system 10 is divided into a first zone 38 and a second zone 40 which, for purposes of illustration, are shown in FIG. 6 as being separated by an imaginary dashed line 42. The first zone 38 has a greater number of the overall plurality of bond points per unit area than the second area 40. In addition, the first zone 38 completely surrounds the second zone 40 thereby creating a system 10 wherein the periphery of the system 10 may have a greater degree of bonding than the central portion of the system 10. In an alternative embodiment, there may be no bond points in the second zone 40. Also, in another embodiment, the periphery of the system 10 may have a lesser degree of bonding than the central portion of the system 10.

Other combinations of bond point patterns may also be used. For example, indicia, logos and other printed matter may be used as the bond pattern to bond the second sheet 12 to the first sheet 14. Thus the bond pattern could be wording such as “KIMBERLY-CLARK” or “KIMGUARD”.

One possible feature of the present invention is that the two sheets of sterilization wrap may be joined to one another with a sufficient amount of bonding so that the two sheets do not separate. To this end, the sterilization wrap 10 may be viewed as having a first exterior surface 44 and a second exterior surface 46 on opposed sides of the system 10. See FIG. 2. It may be advantageous if the surface area of the bond points does not occupy more than about 50 percent of the surface area of either the first or second exterior surfaces 44 and 46 of the sterilization wrap 10. Other embodiments in the present invention contemplate bond points that occupy less than about 30 percent of the surface area of either the first or second exterior surfaces 44 and 46; less than about 20 percent of the surface area of either the first or second exterior surfaces 44 and 46; less than about 10 percent of the surface area of either the first or second exterior surfaces 44 and 46; and less than about 5 percent of the surface area of either the first or second exterior surfaces 44 and 46.

In one embodiment, the two sheets of wrap may be sufficiently joined to one another so that they do not readily separate from one another throughout the process of removing the sterilization wrap from its original packaging, wrapping the items to be sterilized with the wrap and unwrapping the sterilized items for use.

The bonded sheets may come in several sizes to wrap various size items and trays. Typical sizes include 18, 24, 30, 36, 40, 45, 48 and 54 inch square wrappers as well as 54×72 inch rectangular wrappers. To wrap an item, in this case a sterilization wrap tray 18 such as shown in FIG. 1, the item is placed on top of the system 10 in contact with the first sheet 14 such that the four corners of the wrap may be folded over onto the package one at a time. Once the folding is completed, the wrap is sealed with tape and the wrapped package is ready to be sterilized.

Each of the sheets may have its own special characteristics. One possible primary function of the first sheet 14 may be to act as a primary filtration barrier while one possible primary function of the second sheet 12 may be to provide strength with a secondary function of also providing a barrier to bacteria and other contaminants.

Both the second sheet 12 and the first sheet 14 may be made from a number of nonwoven materials. The nonwoven materials may be made from either or both natural and synthetic fibers such as paper, fibrous polymeric nonwovens, as well as films which are capable of passing sterilants and retarding transmission of bacteria and other contaminants.

Nonwoven sterilization wraps have become particularly well-liked due to their barrier properties, economics and consistent quality. The nonwoven materials may be made from a variety of processes including, but not limited to, air laying processes, wet laid processes, hydroentangling processes, spunbonding, meltblowing, staple fiber carding and bonding, and solution spinning. The fibers themselves may be made from a variety of both natural and synthetic materials including, but not limited to, cellulose, rayon, polyesters, polyolefins and many other thermoplastic materials. The fibers may be relatively short, staple length fibers, typically less than 3 inches, or longer more continuous fibers such as are produced by spunbonding and meltblowing processes. Whatever materials are chosen, the resultant wrap may be selected to be compatible with the particular sterilization technique being used and to provide both strength and barrier properties to maintain the sterile nature of the wrapped contents until use.

It has been found that polyolefin-based fibers and their resultant nonwovens are particularly well-suited for the production of sterilization wrap. Polypropylene spunbond nonwovens, such as are produced by the Assignee of record, Kimberly-Clark Worldwide, Inc., may be used to impart strength characteristics to the sterilization wrap and in particular, the second sheet 12. In more refined embodiments, the second sheet 12 may be made from laminates, such as a laminate of spunbond and meltblown or spunbond, meltblown, spunbond to impart both strength and barrier properties to the second sheet 12.

A spunbond, meltblown, spunbond material is made from three separate layers which are laminated to one another. The method of making these layers is known and described in commonly assigned U.S. Pat. No. 4,041,203 to Brock et al., which is incorporated herein in its entirety by reference. The material of Brock et al. is a three layer laminate of spunbond/meltblown/spunbond that is also commonly referred to by the acronym “SMS”. The two outer layers of SMS are a spunbond material made from extruded polyolefin fibers laid down in a random pattern and then bonded to one another. The inner layer is a meltblown layer also made from extruded polyolefin fibers that may have a smaller diameter and sometimes having a more discontinuous length than the fibers in the spunbonded layers. As a result, the meltblown layer provides increased barrier properties due to it fine fiber structure which permits the sterilizing agent to pass through the fabric while preventing passage of bacteria and other contaminants. Conversely, the two outer spunbond layers provide a greater portion of the strength factor in the overall laminate.

The laminate may be prepared using an intermittent bond point pattern that is employed with the pattern being substantially regularly repeating over the surface of the laminate. The pattern is selected such that the bond points occupy about 5 to about 50% of the surface area of the laminate. In an alternative embodiment, the bond points occupy about 10 to about 30% of the surface area of the laminate.

A particular feature of the present invention is the selected tailoring available for each of the layers in the respective second sheet 12 and first sheet 14. While the two sheets may be identical to one another, in alternative embodiments of the present invention the second sheet 12 may be designed to have higher strength properties than the first sheet 14. This is to provide a stronger barrier to tears and other possible breaches of the wrapped item from exterior objects. Conversely, in other embodiments of the present invention, the first sheet 14 may be designed to have higher barrier properties than the second sheet 12. Adjusting the barrier and strength properties may be accomplished by adjusting the basis weights of the first and second sheets, as well as the basis weights of each of the individual layers within each of the sheets. Suitable basis weight ranges for either of the sheets may range between about 0.5 and about 3.5 ounces per square yard (osy).

One particular example of a single step sterilization wrap comprises a second sheet made from a strength barrier web laminate and a first sheet made from a barrier web laminate with the strength barrier web laminate and the barrier web laminate being placed adjacent to one another in substantially face-to-face or superimposed relationship with the laminates being joined to one another at one or more bond points. Each of the layers may be made from a spunbond/meltblown/spunbond laminate as taught, for example, by U.S. Pat. 4,041,203. Thus the strength barrier web laminate may comprise a first strength layer made from randomly deposited fibers, a second strength layer made from randomly deposited fibers and an intermediate barrier layer made from randomly deposited fibers with the fibers in the intermediate barrier layer having an average fiber diameter which is less than the average fiber diameter of the fibers in either of the first or second strength layers. In addition, the intermediate barrier layer is disposed between and bonded to the first and second reinforcing layers. This strength barrier web laminate may form the second sheet 12. The first sheet 14 may be made from a barrier web laminate comprising a third strength layer made from randomly deposited fibers and a fourth strength layer made from randomly deposited fibers with a second intermediate barrier layer made from randomly deposited fibers. Here again the fibers of the second intermediate barrier layer have an average fiber diameter which is less than the average fiber diameter of either the third or fourth strength layers and the second intermediate barrier layer is disposed between and bonded to the third and fourth strength layers. To provide added strength, the second sheet comprised of the strength barrier web laminate may have a greater grab tensile strength than the first sheet and the first sheet made from the barrier web laminate may have a dry spore penetration rate which is lower than the second sheet and a bacterial filtration efficiency which is greater than the second sheet.

When designing first and second sheets with different properties, it may be important that system 10 be positioned such that proper sterilization wrap surface faces the item to be wrapped and the other wrap surface faces away from the wrapped item. Typically this will mean that the first sheet 14 is in contact with the item 18 to be wrapped and the second sheet 12 will be positioned away from the wrapped item 18.

To demonstrate the attributes of the present invention, several sterilization wraps 10 were prepared and then tested against other currently available sterilization wraps.

The products evaluated were:

KIMGUARD ONE-STEP® Heavy-Duty Sterilization Wrap (Kimberly Clark, 48″×48″, Lot T01/19/00-12:53 REF 62148, *+H4186214817)

SIMUL-WRAP® Sterilization Wrap (ATI, 45″×45″, Lot 0276312, Grade 33545)

In the present example, the tests were formatted using a single-sheet, a double-sheet, and a double-sheet with one sheet rotated 90 degrees. The testing was performed using 10 sample reps per test and tested MD and CD Tensile Strengths using a Strip and Grab test and tested MD and CD Tear using a Trapezoid test.

TABLE 1

| | | | | | | | | | CD Trap | | MD Trap | | | | |
|---|---|---|---|---|---|---|---|---|---|---|---|---|---|---|---|
| | CD Strip | | MD Strip | | CD Grab | | MD Grab | | Tear: | | Tear: | | | | |
| | Tensile: | | Tensile: | | Tensile: | | Tensile: | | 1^{st} & | | 1^{st} & | | | | |
| | Peak Load | | Peak Load | | Peak Load | | Peak Load | | High Peak | | High Peak | | | | |
| | (lbs) | | (lbs) | | (lbs.) | | (lbs.) | | (lbs.) | | (lbs.) | | Strip Tensile: | Grab Tensile: | Trap Tear: |
| Sample ID | AVG | SD | AVG | SD | AVG | SD | AVG | SD | AVG | SD | AVG | SD | MD-CD Ratio | MD-CD Ratio | MD-CD Ratio |
| KGHD Single Sheet | 33.4 | 2.0 | 39.7 | 2.2 | 33.2 | 2.6 | 33.0 | 2.5 | 9.4 | 1.7 | 10.0 | 1.0 | 1.2 | 1.0 | 1.1 |
| KGHD One-Step | 65.7 | 2.1 | 81.6 | 3.1 | 63.5 | 3.8 | 68.2 | 4.4 | 16.6 | 1.9 | 17.9 | 3.3 | 1.2 | 1.1 | 1.1 |
| KGHD One-Step (90 deg) | 71.6 | 2.2 | 75.7 | 2.9 | 57.3 | 3.8 | 66.4 | 4.2 | 17.1 | 1.9 | 17.3 | 2.7 | 1.1 | 1.0 | 1.0 |
| ATI Single Sheet | 19.6 | 1.0 | 36.0 | 1.0 | 19.7 | 1.2 | 31.3 | 1.5 | 8.4 | 1.0 | 15.3 | 1.4 | 1.8 | 1.6 | 1.8 |
| ATI Simul-Wrap | 38.9 | 0.6 | 74.5 | 3.1 | 41.2 | 1.7 | 64.4 | 3.5 | 13.0 | 1.1 | 9.9 | 3.6 | 1.9 | 1.6 | 2.3 |
| ATI Simul-Wrap (90 deg) | 58.3 | 2.9 | 56.2 | 2.2 | 51.7 | 2.5 | 49.7 | 2.0 | 20.3 | 1.5 | 22.0 | 3.0 | 1.0 | 1.0 | 1.1 |

As shown in Table 1, current products have MD-oriented strength and tear properties. The higher the speed of manufacturing may result in an even higher MD fiber alignment.

In looking at MD-CD ratios, strength and tear properties became more isotropic in the two layer products with 90-degree “sheet-to-sheet” product construction. By rotating the orientation of the sheets, more fibers in one of the sheets became substantially orthogonal to the fibers in the other sheet, therefore increasing CD strength and tear resistance.

Having thus described the invention in detail, it should be apparent that various modifications and changes may be made without departing from the spirit and scope of the present invention. For example, a wide variety of individual sterilization wraps have been described herein. Thus, a wide variety of combinations of first and second sheets are possible including combinations of both disposable and reusable sterilization wraps. The first and second sheets may be made from the same or different basis weight materials to engineer selected properties into each of the wraps. In addition, a wide variety of bonding techniques were also disclosed which may be used alone or in combination with each other to impart varying bond point patterns to the sterilization wrap of the present invention. Consequently, these and other modifications are contemplated to be within the spirit and scope of the following claims.

We claim:

1. A single-step sterilization wrap comprising:
a first sheet comprising a nonwoven material having a majority of fibers oriented in a direction parallel, or substantially parallel, to one side of the sheet;
a second sheet comprising a nonwoven material having a majority of fibers oriented in a direction parallel, or substantially parallel, to one side of the sheet;
wherein the second sheet is rotated and joined to the first sheet at one or more bond points such that the majority of oriented fibers in the second sheet are substantially orthogonal to the majority of oriented fibers in the first sheet.

2. The sterilization wrap of claim 1, wherein the first sheet is formed from a first spunbond/meltblown/spunbond laminate, and the second sheet is formed from a second spunbond/meltblown/spunbond laminate.

3. The sterilization wrap of claim 1, wherein the sterilization wrap has a first exterior surface and a second exterior surface each defining a respective surface area and wherein the bond points occupy no more than 50% of the surface area of either the first or second exterior surfaces of the sterilization wrap.

4. The sterilization wrap of claim 3, wherein the sterilization wrap has a first exterior surface and a second exterior surface each defining a respective surface area and wherein the bond points occupy no more than 30% of the surface area of either the first or second exterior surfaces of the sterilization wrap.

5. The sterilization wrap of claim 4, wherein the sterilization wrap has a first exterior surface and a second exterior surface each defining a respective surface area and wherein the bond points occupy no more than 20% of the surface area of either the first or second exterior surfaces of the sterilization wrap.

6. The sterilization wrap of claim 5, wherein the sterilization wrap has a first exterior surface and a second exterior surface each defining a respective surface area and wherein the bond points occupy no more than 10% of the surface area of either the first or second exterior surfaces of the sterilization wrap.

7. The sterilization wrap of claim 6, wherein the sterilization wrap has a first exterior surface and a second exterior surface each defining a respective surface area and wherein the bond points occupy no more than 5% of the surface area of either the first or second exterior surfaces of the sterilization wrap.

8. The sterilization wrap of claim 1, wherein the sterilization wrap defines a first zone and a second zone, the first zone having a greater number of the bond points per unit area than the second zone.

9. The sterilization wrap of claim 8, wherein the second zone is surrounded by the first zone.

10. The sterilization wrap of claim 9, wherein the second zone is located about the periphery of the sterilization wrap and the first zone has no bond points.

11. The sterilization wrap of claim 8, wherein the first zone is surrounded by the second zone.

12. The sterilization wrap of claim 1, wherein the first sheet and the second sheet each have a basis weight of from about 0.5 and about 3.5 ounces per square yard.

13. The sterilization wrap of claim 1, wherein the second sheet is rotated to about 90 degrees relative to the first sheet.

14. The sterilization wrap of claim 1, wherein the second sheet is rotated from about 80 to about 90 degrees relative to the first sheet.

15. The sterilization wrap of claim 1, wherein the second sheet is rotated from about 70 to about 90 degrees relative to the first sheet.

16. The sterilization wrap of claim 1, wherein the second sheet is rotated from about 60 to about 90 degrees relative to the first sheet.

17. A single-step sterilization wrap comprising:
a first sheet comprising a spunbond/meltblown/spunbond laminate having a majority of fibers oriented in a direction parallel, or substantially parallel, to one side of the sheet;
a second sheet comprising a spunbond/meltblown/spunbond laminate having a majority of fibers oriented in a direction parallel, or substantially parallel, to one side of the sheet;
wherein the first sheet is joined to the second sheet at one or more bond points such that the majority of oriented fibers in the second sheet are substantially orthogonal to the majority of oriented fibers in the first sheet.

18. The sterilization wrap of claim 17, wherein the sterilization wrap has a first exterior surface and a second exterior surface each defining a respective surface area and wherein the bond points occupy no more than 50% of the surface area of either the first or second exterior surfaces of the sterilization wrap.

19. The sterilization wrap of claim 18, wherein the sterilization wrap has a first exterior surface and a second exterior surface each defining a respective surface area and wherein the bond points occupy no more than 30% of the surface area of either the first or second exterior surfaces of the sterilization wrap.

20. The sterilization wrap of claim 19, wherein the sterilization wrap has a first exterior surface and a second exterior surface each defining a respective surface area and wherein the bond points occupy no more than 20% of the surface area of either the first or second exterior surfaces of the sterilization wrap.

21. The sterilization wrap of claim 20, wherein the sterilization wrap has a first exterior surface and a second exterior surface each defining a respective surface area and wherein the bond points occupy no more than 10% of the surface area of either the first or second exterior surfaces of the sterilization wrap.

22. The sterilization wrap of claim 21, wherein the sterilization wrap has a first exterior surface and a second exterior surface each defining a respective surface area and wherein the bond points occupy no more than 5% of the surface area of either the first or second exterior surfaces of the sterilization wrap.

23. The sterilization wrap of claim 17, wherein the sterilization wrap defines a first zone and a second zone, the first zone having a greater number of the bond points per unit area than the second zone.

24. The sterilization wrap of claim 23, wherein the second zone is surrounded by the first zone.

25. The sterilization wrap of claim 24, wherein the second zone is located about the periphery of the sterilization wrap and the first zone has no bond points.

26. The sterilization wrap of claim 23, wherein the first zone is surrounded by the second zone.

27. The sterilization wrap of claim 17, wherein the first sheet and the second sheet each have a basis weight of from about 0.5 and about 3.5 ounces per square yard.

28. The sterilization wrap of claim 17, wherein the second sheet is rotated from about 60 to about 90 degrees relative to the first sheet.
